(19) United States
(12) Patent Application Publication
(10) Pub. No.: US 2017/0124701 A1
(43) Pub. Date: May 4, 2017

(54) SYSTEM AND METHOD FOR MEASURING ARTERY THICKNESS USING ULTRASOUND IMAGING

(71) Applicant: Arizona Board of Regents on behalf of Arizona State University, Scottsdale, AZ (US)

(72) Inventors: Jianming Liang, Scottsdale, AZ (US); Haripriya Sharma, Tempe, AZ (US); Ramsri G. Golla, Sunnyvale, CA (US); Yu Zhang, Tempe, AZ (US); Christopher B. Kendall, Phoenix, AZ (US); Robert Todd Hurst, Scottsdale, AZ (US); Nima Tajbakhsh, Tempe, AZ (US)

(21) Appl. No.: 15/126,600

(22) PCT Filed: Mar. 17, 2015
(86) PCT No.: PCT/US2015/020908

§ 371 Date: Sep. 16, 2016

Related U.S. Application Data
(60) Provisional application No. 61/954,386, filed on Mar. 17, 2014.

(51) Int. Cl. G06T 7/00; A61B 8/00; G06T 7/11; A61B 8/08

(52) U.S. Cl. CPC G06T 7/0012; A61B 8/0891; A61B 8/0858; A61B 8/463; G06T 7/11; G06T 2207/30101; G06T 2207/10132

(57) ABSTRACT

A system and method for automating the selection of end-diastolic ultrasound frames (EUFs] and regions of interest (ROIs] of the common carotid artery (CCA] to measure the carotid intima-media thickness (CIMT] is provided. The EUFs are selected based on the QRS complex of the ECG signal associated with an ultrasound video, and the ROI is detected based on image intensity and curvature of the carotid artery bulb. The CIMT and a vascular age of a patient is calculated and displayed on a report.

### CROSS-REFERENCE TO RELATED APPLICATIONS

This application claims the benefit of the filing date of U.S. provisional patent application Ser. No. 61/954,386 entitled “SYSTEM AND METHOD FOR MEASURING ARTERY THICKNESS USING ULTRASOUND IMAGING” filed Mar. 17, 2014, the entire contents of which are incorporated by reference herein for all purposes.

### STATEMENT REGARDING FEDERALLY SPONSORED RESEARCH

N/A

### BACKGROUND

The subject matter described herein relates to systems and methods for analyzing carotid artery intima-media thickness (CIMT). More particularly, the subject matter relates to a system and method for automatically selecting end-diastolic ultrasound frames (EUFs) and determining regions of interest (ROIs) in ultrasound videos to screen for arterial pathology consistent with advanced atherosclerosis.

The CIMT technique is a noninvasive ultrasound test to investigate for sub-clinical atherosclerosis in patients for cardiovascular disease (CVD) risk assessment. CIMT is measured based on ROIs in the cardiac cycle timing at EUFs. In addition, increased CIMT may be an independent predictor of future cardiovascular events, including heart attacks, cardiac death, and stroke. In a CIMT exam, a high-resolution B-mode ultrasound transducer is applied on the patient's neck to image the common carotid artery (CCA). A sonographer manually selects the EUF of interest from the captured ultrasound video, and searches within each of the selected frames for the ROI where the combined thickness of intimal and medial layers of the CCA walls can be measured reliably. However, the manual selection of the EUFs and ROIs can be a tedious and time consuming process that demands specialized expertise and experience.

Published studies on CIMT measurements in animals and humans of varying ages have made it possible to develop a reference quartile range of progression of CIMT for “normal” and pathologic at different ages. Typically, the arterial intimal-medial thickness tends to increase with the age of the patient, and if present chronicity and intensity of risk factors for atherosclerosis. After the measurements are taken, the results are compared against the reference range and a report indicating the status of “vascular age” is generated. If the vascular age and quartile matches the chronological age or younger, then the patient is said to have no evidence of sub-clinical atherosclerosis and can be placed at a lower risk for the possibility of future cardiovascular events. However, if the vascular age and quartile is greater than the chronological age reference range values, the patient is said to have evidence of sub-clinical atherosclerosis and can be vulnerable to increased possibility of future CVDs and therefore precautionary measures should be taken.

As previously described, measurement of CIMT and estimation of vascular age can be a tedious task. The accuracy and speed of CIMT measurement and estimation often varies depending on the users' experience and level of expertise. In addition, inadequate familiarity can prolong the reading time of ultrasound videos, thus leading to increased human efforts and decreased performance.

Therefore, there is a need for systems and methods to automatically and/or semi-automatically select EUFs and determine ROIs in ultrasound videos to provide a more user-friendly and less time consuming solution to interpret CIMT measurements.

### SUMMARY

The present disclosure describes embodiments that overcome the aforementioned drawbacks by providing a system and method that reduces CIMT interpretation time by automatically selecting EUFs and determining ROIs in ultrasound videos. EUFs are selected based on the QRS complex of the electrocardiogram (ECG) signal associated with the ultrasound video, and the ROI is detected based on image intensity and curvature of the carotid artery bulb. Once an EUF is selected and its corresponding ROI is determined, the system measures CIMT using active contour models (i.e., the snake algorithm) extended with hard constraints by computing the average thickness and maximum thickness. The vascular age may then be calculated and a patient report may be generated.

In accordance with one aspect, a method for automatically selecting ultrasound frames and regions of interest of an artery of a subject includes acquiring an imaging data set from a portion of the subject including the artery. A look up table is generated to map a plurality of ultrasound frames to a location in an electrocardiogram (ECG) signal. The imaging dataset is processed to identify, using the look up table, the plurality of ultrasound frames. The regions of interest of the artery are detected by identifying a region of the artery defined by artery edges. Using an algorithm, a thickness of the artery is calculated using the identified plurality of ultrasound frames and regions of interest of the artery. A report is generated related to the thickness of the artery of the subject.

In accordance with another aspect, a system for automatically selecting ultrasound frames and regions of interest of an artery of a subject is provided. The system includes an imaging data set acquired from a portion of the subject including the artery. A look up table is provided to map a plurality of ultrasound frames to a location in an electrocardiogram (ECG) signal. A processor is configured to process the imaging dataset to identify, using the look up table, the plurality of ultrasound frames. The processor is further configured to detect the regions of interest of the artery by identifying a region of the artery defined by artery edges and calculate, using an algorithm, a thickness of the artery using the identified plurality of ultrasound frames and regions of interest of the artery to generate a report related to the thickness of the artery of the subject.

The foregoing and other aspects and advantages of the disclosure will appear from the following description. In the description, reference is made to the accompanying drawings which form a part hereof, and in which there is shown by way of illustration one embodiment. Such embodiment does not necessarily represent the full scope of the disclosure, however, and reference is made therefore to the claims and herein for interpreting the scope of the disclosure.

### BRIEF DESCRIPTION OF THE DRAWINGS

FIG. 1 is a block diagram of an exemplary ultrasonic imaging system;

FIG. 2 is a block diagram of a receiver that forms part of the exemplary system of FIG. 1;

FIG. 3 is a flow chart setting forth the steps of processes for automatically selecting EUFs and determining ROIs in ultrasound videos to interpret CIMT measurements using the exemplary system of FIG. 1;

FIG. 4 is a screen shot of an exemplary user interface used to display the CIMT measurements and regions of interest;

FIG. 5 is a screen shot of an exemplary reconstructed ECG signal by superimposition of difference signals;

FIG. 6A is an exemplary image showing a detected artery region (AR) with reference line l;

FIG. 6B is an intensity plot showing detected local minima that indicates the location of the reference line l passing through the AR of FIG. 6A;

FIG. 6C is a refined edge map of FIG. 6A showing edges of the detected AR and reference line l on the detected artery corresponding to the local minima of FIG. 6B;

FIG. 6D is an exemplary user interface showing a horizontal sliding window w that is centered along the reference line l of the refined edge map of FIG. 6C;

FIG. 6E is an exemplary user interface showing upper and lower boundaries of the artery traced nearest to the reference line l of FIG. 6D and the sliding window w with the highest total curvature value selected and a detected ROI; and

FIG. 6F is an exemplary user interface showing a comparison of a bulb region of the artery and the detected ROI of the artery of FIG. 6E.

### DETAILED DESCRIPTION

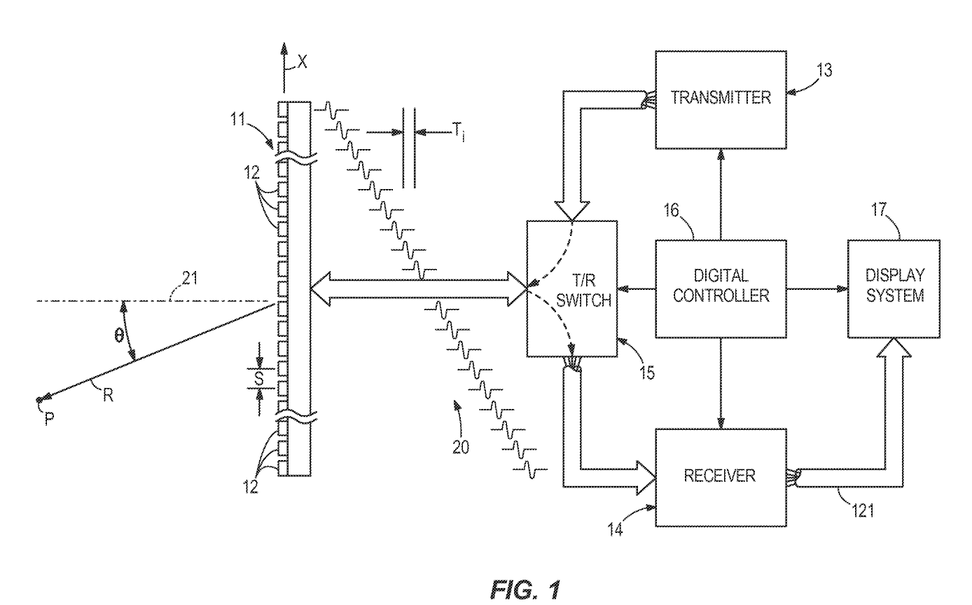

Referring particularly to FIG. 1, an exemplary ultrasonic imaging system includes a transducer array 11 comprised of a plurality of separately driven elements 12 that each produce a burst of ultrasonic energy when energized by a pulse produced by a transmitter 13. The ultrasonic energy reflected back to the transducer array 11 from the subject under study is converted to an electrical signal by each transducer element 12 and applied separately to a receiver 14 through a set of switches 15. The transmitter 13, receiver 14 and the switches 15 are operated under the control of a digital controller 16 responsive to the commands input by the human operator. A complete scan is performed by acquiring a series of echoes in which the switches 15 are set to their transmit position, the transmitter 13 is gated on momentarily to energize each transducer element 12, the switches 15 are then set to their receive position, and the subsequent echo signals produced by each transducer element 12 are applied to the receiver 14. The separate echo signals from each transducer element 12 are combined in the receiver 14 to produce a single echo signal that is employed to produce a line in an image on a display system 17.

The transmitter 13 drives the transducer array 11 such that the ultrasonic energy produced is directed, or steered, in a beam or pulse. A B-scan can therefore be performed by moving this beam through a set of angles from point-to-point rather than physically moving the transducer array 11. To accomplish this, the transmitter 13 imparts a time delay (Ti) to the respective pulses 20 that are applied to successive transducer elements 12. If the time delay is zero (Ti=0), all the transducer elements 12 are energized simultaneously and the resulting ultrasonic beam is directed along an axis 21 normal to the transducer face and originating from the center of the transducer array 11. As the time delay (Ti) is increased, the ultrasonic beam is directed downward from the central axis 21 by an angle θ. A sector scan is performed by progressively changing the time delays Ti in successive excitations. The angle θ is thus changed in increments to steer the transmitted beam in a succession of directions.

Referring still to FIG. 1, the echo signals produced by each burst of ultrasonic energy emanate from reflecting objects located at successive positions (R) along the ultrasonic beam. These are sensed separately by each element 12 of the transducer array 11 and a sample of the magnitude of the echo signal at a particular point in time represents the amount of reflection occurring at a specific range (R). Due to the differences in the propagation paths between a focal point P and each transducer element 12, however, these echo signals will not occur simultaneously and their amplitudes will not be equal. The function of the receiver 14 is to amplify and demodulate these separate echo signals, impart the proper time delay to each and sum them together to provide a single echo signal that accurately indicates the total ultrasonic energy reflected from each focal point P located at range R along the ultrasonic beam oriented at the angle θ.

To simultaneously sum the electrical signals produced by the echoes from each transducer element 12, time delays are introduced into each separate transducer element channel of the receiver 14. In the case of the linear transducer array 11, the delay introduced in each channel may be divided into two components, one component is referred to as a beam steering time delay, and the other component is referred to as a beam focusing time delay. The beam steering and beam focusing time delays for reception are precisely the same delays (Ti) as the transmission delays described above. However, the focusing time delay component introduced into each receiver channel is continuously changing during reception of the echo to provide dynamic focusing of the received beam at the range R from which the echo signal emanates.

Under the direction of the digital controller 16, the receiver 14 provides delays during the scan such that the steering of the receiver 14 tracks with the direction of the beam steered by the transmitter 13 and it samples the echo signals at a succession of ranges and provides the proper delays to dynamically focus at points P along the beam. Thus, each emission of an ultrasonic pulse results in the acquisition of a series of data points that represent the amount of reflected sound from a corresponding series of points P located along the ultrasonic beam.

The display system 17 receives the series of data points produced by the receiver 14 and converts the data to a form producing the desired image. For example, if an A-scan is desired, the magnitude of the series of data points is merely graphed as a function of time. If a B-scan is desired, each data point in the series is used to control the brightness of a pixel in the image, and a scan comprised of a series of measurements at successive steering angles (θ) is performed to provide the data necessary for display of an image.

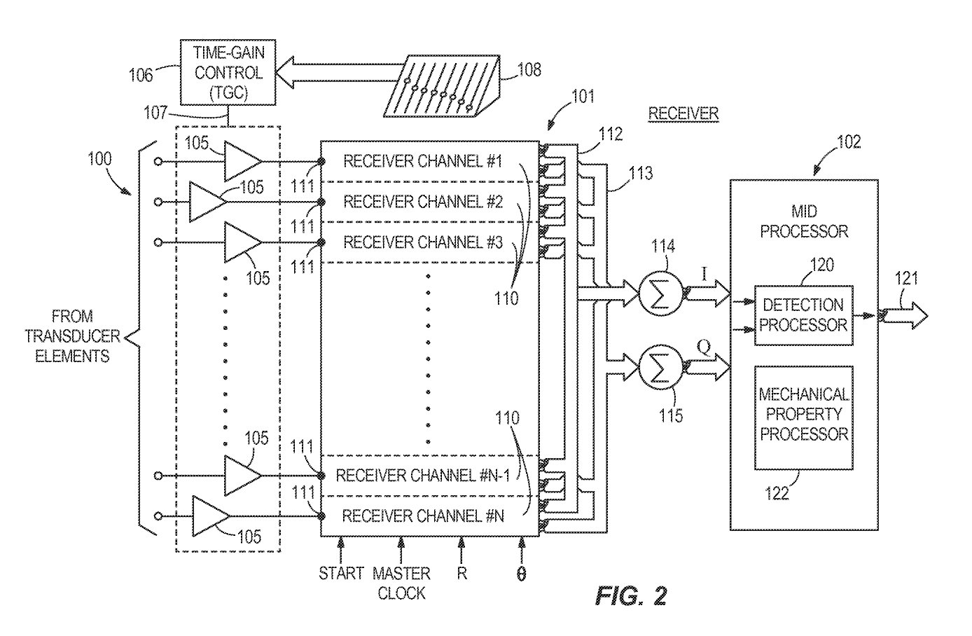

Referring particularly to FIG. 2, the receiver 14 is comprised of three sections: a time-gain control section 100, a beam forming section 101, and a mid processor 102. The time-gain control section 100 includes an amplifier 105 for each of the N=128 receiver channels and a time-gain control circuit 106. It is noted that 128 receiver channels is selected for exemplary purposes and that other numbers of channels are contemplated. The input of each amplifier 105 is connected to a respective one of the transducer elements 12 to receive and amplify the echo signal that it receives. The amount of amplification provided by the amplifiers 105 is controlled through a control line 107 that is driven by the time-gain control circuit 106. As is well known in the art, as the range of the echo signal increases, its amplitude is diminished. As a result, unless the echo signal emanating from more distant reflectors is amplified more than the echo signal from nearby reflectors, the brightness of the image diminishes rapidly as a function of range (R). This amplification is controlled by the operator who manually sets time gain compensation (TGC) linear potentiometers 108 to values that provide a relatively uniform brightness over the entire range of the sector scan. The time interval over which the echo signal is acquired determines the range from which it emanates, and this time interval is divided by the TGC control circuit 106. The settings of the potentiometers are employed to set the gain of the amplifiers 105 during each of the respective time intervals so that the echo signal is amplified in ever increasing amounts over the acquisition time interval.

The beam forming section 101 of the receiver 14 includes separate receiver channels 110. Each receiver channel 110 receives the analog echo signal from one of the TGC amplifiers 105 at an input 111, and it produces a stream of digitized output values on an “I” bus 112 and a “Q” bus 113. Each of these I and Q values represents a sample of the echo signal envelope at a specific range (R). These samples have been delayed in the manner described above such that when they are summed at summing points 114 and 115 with the I and Q samples from each of the other receiver channels 110, they indicate the magnitude and phase of the echo signal reflected from a point P located at range R on the steered beam (θ).

Referring still to FIG. 2, the mid processor section 102 receives the beam samples from the summing points 114 and 115. The I and Q values of each beam sample is a 16-bit digital number that represents the in-phase and quadrature components of the magnitude of the reflected sound from a point (R,θ). The mid processor 102 can perform a variety of calculations on these beam samples, where choice is determined by the type of image to be reconstructed.

For example, a conventional ultrasound image may be produced by a detection processor 120 that calculates the magnitude M of the echo signal from its I and Q components:

M=\sqrt{I²+Q²}. (1)

The resulting magnitude values output at 121 to the display system 17 result in an image in which the magnitude of the reflected echo at each image pixel is indicated.

This embodiment is implemented by a mechanical property processor 122 that forms part of the mid-processor 102. As will be explained in detail below, this processor 102 receives the I and Q beam samples acquired during a sequence of measurements of the subject tissue (i.e., artery) and calculates a mechanical property (i.e., thickness) of the tissue.

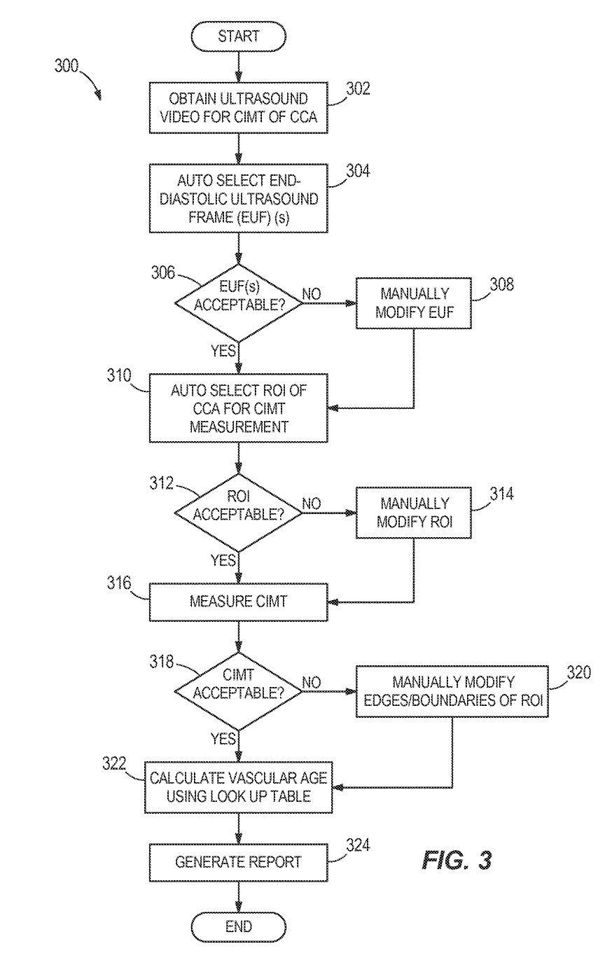

Referring now to FIG. 3, a flow chart is provided setting forth exemplary steps 300 of a method to reduce CIMT interpretation time by automatically selecting EUFs and determining ROIs in ultrasound videos in accordance with one embodiment. To begin the process, an imaging data set, such as an ultrasound video, for determining the CIMT of the CCA of a patient may be obtained at process block 302. The ultrasound video may be obtained from an ultrasound system, such as the ultrasound system shown in FIGS. 1 and 2. More specifically, the ultrasound system may be a B-Mode ultrasound system using an 8-14 MHz linear array transducer. The ultrasound system is configured to image the CCA, for example, of the patient using a systematic imaging protocol.

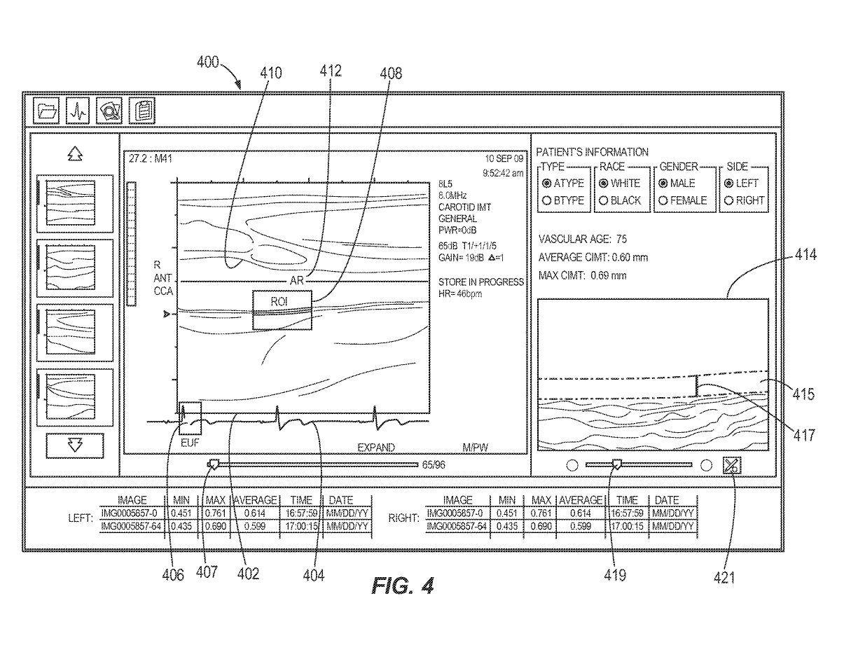

At process block 304, EUFs are detected automatically from the acquired ultrasound video at process block 302 for CIMT measurement and analysis. The EUF detection may be based on an electrocardiogram, for example. Typically, the ultrasound test for CIMT is performed with electrocardiography. To establish correspondences between imaging and electrocardiography data, a user interface 400, as shown in FIG. 4, may display an ECG signal 404 at the bottom of each ultrasound frame 402 of the user interface 400. The ECG signal 404 may include two cine-loops of three beats and three separate end-diastole phases. A cardiac cycle indicator 406 in the ECG signal 404 signifies when, during a cardiac cycle, the ultrasound frame 402 has been captured. Because the frame of interest is to be selected close to the end of the diastolic phase, the positions of the QR waves in the ECG signal 404 can be used as an indication to localize the target frame. Thus, a lookup table (LUT) that can map each ultrasound frame 402 to a location in the ECG signal 404 is used to select the frames of interest.

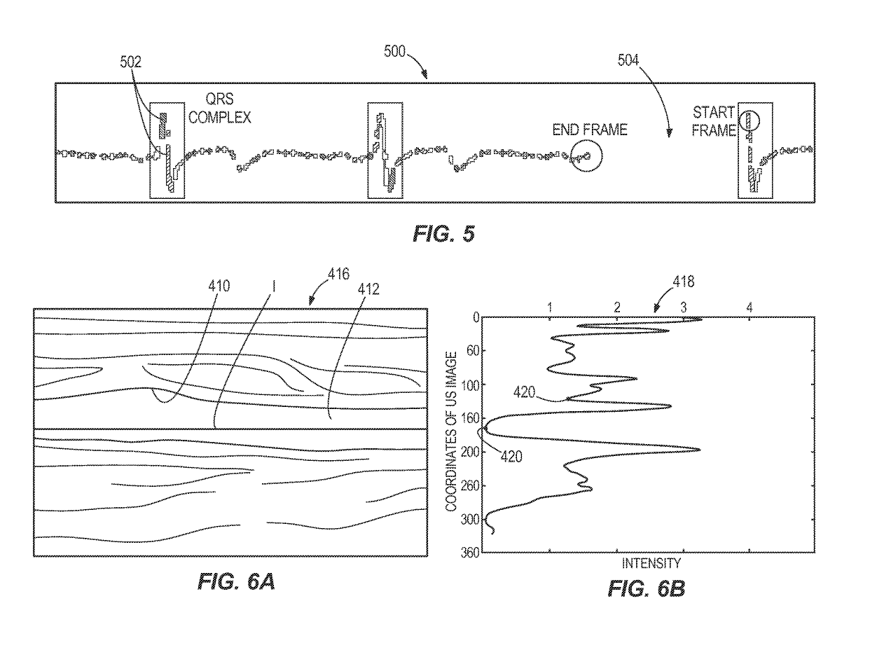

The LUT may be generated by subtracting every two consecutive ultrasound frames 402 and indexing a resultant edge segment with the corresponding frame number. Given two frames 402 captured at time t and t+1, the subtraction image contains a small curvelet from the ECG signal 404, which had been masked out by the cardiac cycle indicator 406 in the frame at time t. The location of each curvelet and the corresponding frame number t may be stored in the lookup table. Repeating this procedure for all consecutive frames results in a number of curvelets, which are further concatenated to form a reconstructed ECG signal 500, as shown in FIG. 5 by different patterns or shades of gray, in which each segment corresponds to a particular ultrasound frame. The reconstructed ECG signal 500 may be formed by superimposition of difference signals obtained from every two consecutive frames for EUF detection, for example. The edge segments shown in patterns 502 or shades of gray, represent the ‘gap’ in the ECG signal for every frame, signifying when, during a cardiac cycle, the ultrasound frame has been captured. The number of these segments corresponds to the number of frames in the ultrasound video. In the reconstructed ECG signal 500, the locations of local maxima (R-waves) are searched for and the system looks into the LUT to identify the frames that correspond to EUFs in the given video. As shown in FIG. 5, the start frame and the end frame of the gap region 504 indicate the segments corresponding to the last and the first frames of the ultrasound video, respectively.

Returning to FIG. 3, at decision block 306, a user, such as a sonographer or physician, can determine whether the automatically detected EUFs, as just described, are acceptable. If the EUFs are not acceptable to the user at decision block 306, the user may manually modify the selected frame 402 at process block 308, for example, by clicking on the frame 402 displayed on the user interface 400 of FIG. 4. Additionally, or alternatively, a slider 407 may be provided on the user interface 400 to navigate the ultrasound frames 402 in the ultrasound video if necessary. However, if the EUFs are acceptable to the user at decision block 306, the system may automatically detect a ROI in the CCA being imaged at process block 310. An example ROI 408 is shown in FIG. 4.

The ROI 408 detected at process block 310 encompasses the segment where the CIMT is to be measured, for example. In one non-limiting example, the ROI 408 may form a rectangle having a length of about 1 cm and a height of about 0.65 cm corresponding to 92 pixels by 60 pixels. The ROI 408 may be identified automatically within the chosen EUF, and include the far wall of the distal 1 cm, for example, of the CCA where the plaques normally develop. As shown in FIG. 4, the ROI 408 may be placed on the intimal and medial layers of the CCA walls, just before the outset of the carotid bulb 410. The carotid bulb 410 is the portion of the CCA where the highest curvature is observed, as shown in FIG. 4. Therefore, to detect the ROI 408, an artery region (AR) 412 is detected and then the curvature along the artery edges may be computed.

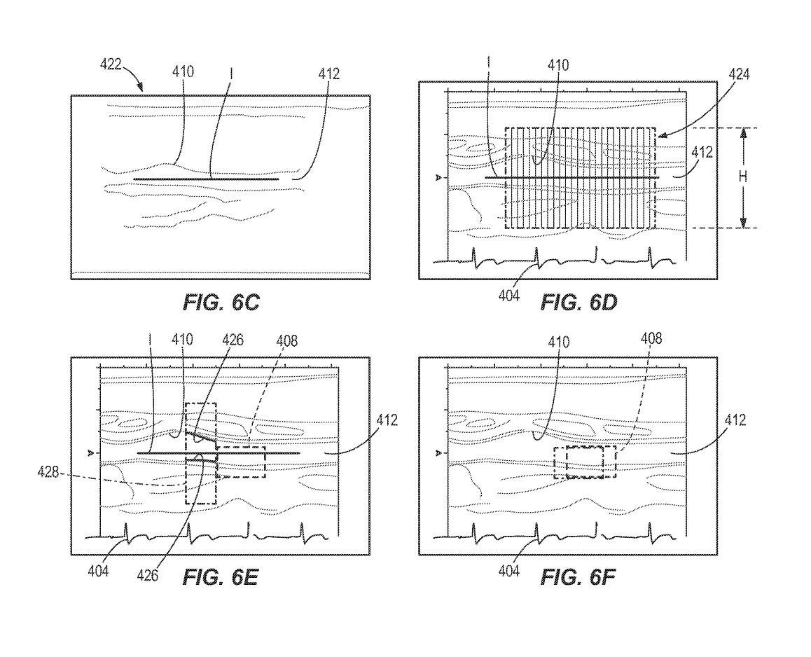

Still referring to FIG. 4, the AR 412 appears black in the ultrasound image displayed on the user interface 400. This property may be utilized to separate out the AR 412 from the rest of the ultrasound image content. To accomplish this, a sliding window (not shown), for example, may be used in a cropped region 416 of the ROI as shown in FIG. 6A, with the width being substantially equal to the width of the cropped region 416 and the height being about 15 pixels, which is the average height of the CCA. The sliding window may be slid down by 1 pixel, for example, and each time an average pixel intensity may be computed. This results in an intensity plot having a 1D signal 418, as shown in FIG. 6B, showing detected local minima 420, which indicates the location of the line l which passes through the AR 412, as best shown in FIG. 6C.

The user interface 400 shown in FIG. 4 may also provide a zoomed-in region 414 of the ROI 408. An overlay 415, which may be a colored overlay, may be provided in the zoomed-in region 414 to show various distances. In one non-limiting example, a first color (e.g., red) shown in the overlay 415 may indicate a larger distance compared to a second color (e.g., green) shown in the overlay 415 which indicates a shorter distance. Additionally, or alternatively, a ruler 417 may be provided to indicate a numerical distance, for example, at a specific location in the zoomed-in region 414. The ruler 417 may be adjusted (i.e., slid) to any location within the zoomed-in region 414 of the ROI 408. A sliding bar 419 may also be provided to control the transparency of the overlay 415. Further, a button 421 may be provided on the user interface 400 to turn the overlay 415 on or off, for example.

Returning again to FIG. 3, once the ROI is detected at process block 310, at decision block 312, a user, such as a sonographer or physician, can determine whether the automatically detected ROI is acceptable. If the ROI is not acceptable to the user at decision block 312, he or she may manually modify the selected ROI at process block 314, for example, by selecting the ROI 408 displayed on the user interface 400 of FIG. 4, and moving to the location as the user desires. However, if the ROI is acceptable to the user at decision block 312, the system may automatically measure CIMT at process block 316. However, in order to measure CIMT, clean segmentation of the CCA may be necessary for reliable curvature estimation.

Thus, the image may be preprocessed by median and Gaussian filtering, for example, and applying canny edge detection techniques to generate an edge map 422, as shown in FIG. 6C. The edge map 422 may then be refined by removing small and unwanted edges through a connected component analysis, for example, that removes connected components that are less than 160 pixels. A horizontal sliding window 424 may be defined, which is centered along reference line l, on the refined edge map, as shown in FIG. 6D. A height H of the sliding window 424, may be triple the average height of the artery, for example, and large enough to encompass the carotid bulb 410.

Referring to FIG. 6D, the window 424 is shown after every 10 pixels for visual purposes. Inside each window 424, artery edges 426 nearest to reference line l may be traced, as shown in FIG. 6E, and the curvature at every pixel on the artery edges 426 is computed. The curvature can be computed by 1/r where r is the radius of curvature. The total curvature of the upper and lower boundary (i.e., the artery edges 426) for the window 424 at each location on the reference line l is determined, and the window 424 with the maximum curvature, represented by rectangle 428 in FIG. 6E, is selected. The highest curvature window 428 depicts the region with the bulb 410. The ROI 408, as mentioned earlier, is placed next to the bulb 410, as shown in FIGS. 6E and 6F.

In some embodiments, the CIMT measurement may performed after the EUF and ROI are determined. The measurement involves Carotid intima-media border detection, CIMT mean, minimum and maximum measurements and vascular age calculation. The work for border detection is a variant of the snake model with hard constraints. The hard constraint mechanisms force the snake model to pass through certain positions or take certain shapes, so that anatomic intricacies can be clearly measured and delineated with simple user interactions. This enables the user to easily adjust the border based on experience and judgment. As previously described, the length of the ROI may be 1 cm, which comprises 92 pixels. The intima-media thickness is the perpendicular distance between the (two borders of the wall) media-adventitia border and the lumen-intima border within the ROI, thereby obtaining 92 lengths corresponding to the 92 pixel points. The mean, maximum and the minimum CIMT from these lengths are then calculated.

Referring once again to FIG. 3, once the CIMT is measured at process block 316, at decision block 318, the user can determine whether the measured CIMT is acceptable. If the measured CIMT is not acceptable to the user at decision block 318, the user may manually modify the edges and/or detected boundaries shown in the zoomed-in region 414 of the ROI 408 of FIG. 4 at process block 320. However, if the measured CIMT is acceptable at decision block 318, the system may calculate a vascular age of the patient at process block 322 using a LUT, such as the Bogalusa Study Database of a given race and gender, for example. Once the vascular age is calculated at process block 322, a corresponding report may be generated at process block 324 and displayed to the user on the display system 17 of FIG. 1. If the calculated vascular age matches the chronological age or is younger than the patient's age, the report may indicate that the patient has a lower risk of heart diseases. However, if the calculated vascular age is older than the chronological age of the patient, the report may indicate that the patient may be vulnerable to CVDs and may recommend precautionary measures to be taken.

Thus, the above described system and method allows for automatic EUF and ROI detection in an ultrasound video for CIMT measurement. The EUFs are selected based on the QRS complex of the ECG signal associated with the ultrasound video, and the ROIs are detected based on image intensity and curvature of the carotid artery bulb. The method for automatic ROI and EUF detection has proven to be fast, reliable, and easy to use. The method is interactive and enables the user to modify the obtained detections. The system and method also reduce user-dependency by automating the CIMT measurement process. Thus, the system and method saves a significant amount of reading time in the process for CIMT measurement, thereby decreasing human efforts when incorporated into ultrasound systems by reducing the effective reading time and user dependency.

The present disclosure has been described in terms of one or more exemplary embodiments, and it should be appreciated that many equivalents, alternatives, variations, and modifications, aside from those expressly stated, are possible and within the scope of the disclosure.

1. A method for automatically selecting ultrasound frames and regions of interest of an artery of a subject, the method comprising:
a) acquiring an imaging data set from a portion of the subject including the artery;
b) generating a look up table to map a plurality of ultrasound frames to a location in an electrocardiogram (ECG) signal;
c) processing the imaging dataset to identify, using the look up table, the plurality of ultrasound frames;
d) detecting the regions of interest of the artery by identifying a region of the artery defined by artery edges;
e) calculating, using an algorithm, a thickness of the artery using the identified plurality of ultrasound frames and regions of interest of the artery; and
f) generating a report related to the thickness of the artery of the subject.

2. The method as recited in claim 1 further comprising calculating a vascular age of the patient based on the measured thickness of the artery, wherein when the vascular age of the patient is above a predetermined threshold, the patient is associated with higher risk of cardiovascular disease.

3. The method as recited in claim 1, wherein identifying the region of the artery defined by the artery edges further includes computing a curvature along the artery edges and identifying a maximum curvature of the artery edges to identify the region of interest.

4. The method as recited in claim 1, wherein acquiring the imaging data set includes acquiring an ultrasound video of the artery of the subject.

5. The method as recited in claim 1, wherein the artery of the patient is a common carotid artery (CCA).

6. The method as recited in claim 1, wherein measuring a thickness of the artery includes measuring a carotid intima-media thickness (CIMT).

7. The method as recited in claim 1, wherein processing the imaging dataset to identify the plurality of ultrasound frames includes automatically selecting end-diastolic ultrasound frames (EUFs) of a common carotid artery (CCA), the EUFs identified based on a QRS complex of the ECG signal corresponding to the imaging dataset.

8. The method as recited in claim 1, wherein processing the imaging dataset to identify the regions of interest of the artery relates to at least one of image intensity and curvature of a carotid artery bulb.

9. The method as recited in claim 1, wherein the algorithm used to measure the thickness of the artery includes computing at least one of an average artery thickness and a maximum artery thickness.

10. The method as recited in claim 1 further comprising providing a user of the imaging dataset an ability to manually modify at least one of the plurality of ultrasound frames, the regions of interest, and the artery edges.

11. A system for automatically selecting ultrasound frames and regions of interest of an artery of a subject, the system comprising:
an imaging data set acquired from a portion of the subject including the artery;
a look up table to map a plurality of ultrasound frames to a location in an electrocardiogram (ECG) signal; and
a processor configured to process the imaging dataset to identify, using the look up table, the plurality of ultrasound frames,
wherein the processor is further configured to detect the regions of interest of the artery by identifying a region of the artery defined by artery edges and calculate, using an algorithm, a thickness of the artery using the identified plurality of ultrasound frames and regions of interest of the artery to generate a report related to the thickness of the artery of the subject.

12. The system as recited in claim 11, wherein the processor is configured to calculate a vascular age of the patient based on the measured thickness of the artery, the vascular age of the patient above a predetermined threshold indicates the patient is associated with higher risk of cardiovascular disease.

13. The system as recited in claim 11, wherein a curvature along the artery edges is computed using the processor to identify the region of interest characterized by a maximum curvature of the artery edges.

14. The system as recited in claim 11, wherein the imaging data set includes an ultrasound video of the artery of the subject.

15. The system as recited in claim 11, wherein the artery of the patient is a common carotid artery (CCA).

16. The system as recited in claim 11, wherein the thickness of the artery includes a carotid intima-media thickness (CIMT).

17. The system as recited in claim 11, wherein the processor is configured to automatically select end-diastolic ultrasound frames (EUFs) of a common carotid artery (CCA) when processing the imaging dataset to identify the plurality of ultrasound frames, the EUFs identified based on a QRS complex of the ECG signal corresponding to the imaging dataset.

18. The system as recited in claim 11, wherein the regions of interest of the artery relates to at least one of image intensity and curvature of a carotid artery bulb.

19. The system as recited in claim 11, wherein the algorithm used to measure the thickness of the artery includes computing at least one of an average artery thickness and a maximum artery thickness.

20. The system as recited in claim 11, wherein the processor is further configured to provide a user of the imaging dataset an ability to manually modify at least one of the plurality of ultrasound frames, the regions of interest, and the artery edges on a user interface.
